(19) United States
(12) Patent Application Publication
(10) Pub. No.: US 2010/0184852 A1
(43) Pub. Date: Jul. 22, 2010

(54) ORGANIC COMPOUNDS FOR TREATMENT OF DISORDERS CONNECTED TO IMPAIRED NEUROTRANSMISSION

(76) Inventors: Antoine De Saizieu, Brunstatt (FR); Regina Goralczyk, Grenzach-Wyhlen (DE); Goede Schueler, Eimeldingen (DE)

Correspondence Address: NIXON & VANDERHYE, PC, 901 NORTH GLEBE ROAD, 11TH FLOOR, ARLINGTON, VA, 22203, US

(21) Appl. No.: 12/376,621

(22) PCT Filed: Aug. 10, 2007
(86) PCT No.: PCT/EP07/07086

§ 371 Date: Nov. 6, 2009

(30) Foreign Application Priority Data
Aug. 11, 2006 (EP) 06016770.7

(51) Int. Cl. A61K 31/365; C07D 307/88; A61P 25/22; A61P 25/00; A61P 25/24; A61P 25/20

(52) U.S. Cl. 514/470; 549/305; 549/310; 549/307

(57) ABSTRACT

The present invention refers to compounds of the general formula (I) wherein R¹ is hydrogen or hydroxy; R² is butyl or butyryl if R¹ is hydroxy, but R² is butyl if R¹ is hydrogen; or R³ and R² taken together are 1-propylidene or 1-butylidene optionally substituted by hydroxy, methyl, or 3-(α, β-dimethylacryloyloxy); the dotted line is an optional bond; X is an optionally substituted aliphatic C4-residue selected from the group consisting of X1, X2, X3, X4, and X5; wherein X is X2, X3 or X5 if the dotted line in formula (I) is absent; and X is X1, X4 or X5 if the dotted line signifies a bond in formula (I) above; R³ and R⁴ are, independently from each other, hydrogen or hydroxy; and R⁵ is hydroxy or butyryl, for use in the treatment of disorders connected to impaired neurotransmission, as well as to dietary and pharmaceutical compositions containing such compounds, and their uses.

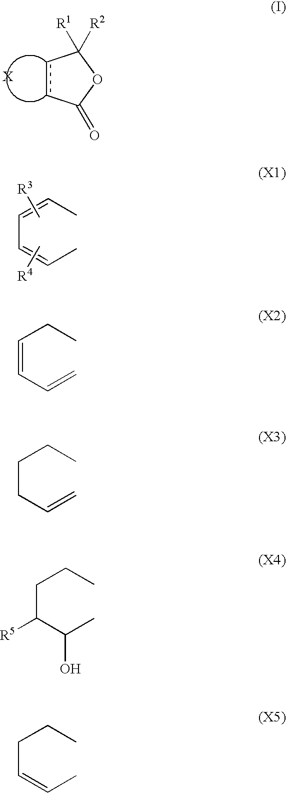

The present invention refers to organic compounds of the general formula I as defined below for use in the treatment of disorders connected to impaired neurotransmission, as well as to dietary and pharmaceutical compositions containing such compounds, and to their uses.

It is well known that impaired neurotransmission, e.g. low neurotransmitter levels, is connected to mental diseases such as depression and generalized anxiety disorders (GAD), and increased susceptibility to stress.

Compounds that increase neurotransmitter levels in the brain and thus enhance their transmission, exhibit therefore antidepressant properties as well as beneficial effects on a variety of other mental disorders (“Neurotransmitters, drugs and brain function” R. A. Webster (ed.), John Wiley & Sons, New York, 2001, p. 187-211, 289-452, 477-498). The main neurotransmitters are serotonin, dopamine, noradrenaline (=norepinephrine), acetylcholine, glutamate, gamma-amino-butyric acid, as well as neuropeptides. Increase in neurotransmission is achieved by increasing the concentration of the neurotransmitter in the synaptic cleft thus making it available for increased or prolonged neurotransmission through inhibition of re-uptake into the pre-synaptic nerve end, or by preventing neuro-transmitter catabolism by inhibition of degrading enzymes such as monoaminooxidase A and B.

Tricyclic antidepressant compounds (TCAs) such as imipramine, amitriptyline, and clomipramine e.g. inhibit the re-uptake of serotonin and noradrenaline. They are widely regarded as among the most effective antidepressants available, but they have a number of disadvantages because they interact with a number of brain receptors, e.g. with cholinergic receptors. Most importantly, TCAs are not safe when taken in overdose, frequently showing acute cardiotoxicity.

Another class of antidepressant drugs are the so-called SSRIs (selective serotonin re-uptake inhibitors) including fluoxetine, paroxetine, sertraline, citalopram, fluvoxamine that block the serotonin transporter (SERT), a high affinity sodium chloride-dependent neuro-transmitter transporter that terminates serotonergic neurotransmission by uptake of serotonin. They have been proven as effective in the treatment of depression and anxiety, and are usually better tolerated than TCAs. These medications are typically started at low dosages and may be increased until they reach a therapeutic level. A common side effect is nausea. Other possible side effects include decreased appetite, dry mouth, sweating, infection, constipation, yawn, tremor, sleepiness and sexual dysfunction.

In addition, compounds that prevent the catabolism of neurotransmitters more broadly by inhibiting the monoaminooxidases (MAOs) A and B exhibit antidepressant effects. MAOs catalyse the oxidation of amino group containing neurotransmitters such as serotonin, noradrenaline, and dopamine.

Furthermore, modulators of neurotransmission exert pleiotropic effects on mental and cognitive functions. These functions differ clearly from those effects reported for food/plant constituents with antioxidant function which exert a neuroprotective effect via reduction of oxidative stress.

In addition, patients often suffer either as a comorbidity to depression, or by itself from generalized anxiety syndrome (GAD). GAD is a highly prevalent anxiety condition and chronic illness in primary care (˜10% of patients) (Wittchen, et al 2005. European Neuropsychopharm. 15:357-376). Patients present themselves to their primary care physician with multiple physical symptoms. GAD is characterized by chronic tension, and anxious worrying and tension (>6 months), which are disabling and uncontrollable, and accompanied by a characteristic hypervigilance syndrome (including restlessness, muscle tension, and sleep problems). If untreated, GAD runs a chronic, fluctuating course and tends to get more severe with age. GAD patients suffer from subsyndromal depression and contribute to the highest overall direct and indirect health economic burden of all anxiety and depressive disorder. Despite high GAD incidence, few sufferers are diagnosed, prescribed medication, or receive psychiatric referral-simple diagnostic tools to aid patient recognition and monitoring are needed. Regardless of specific diagnosis, physicians require effective GAD-symptom treatments. SSRIs such as paroxetine are effective for GAD treatment [Stocchi et al. 2003 J Clin Psych, 63(3):250-258]. Also, systematic reviews and placebo-controlled RCTs (Randomized Clinical Trials) indicate that some SSRIs (escitalopram, paroxetine and sertraline), the SNRI (Selective Norepinephrine Reuptake Inhibitors) venlafaxine, some benzodiazepines (alprazolam and diazepam), the tricyclic imipramine, and the 5-HT1A partial agonist buspirone are all efficacious in acute treatment. In general, the effect of treatment is often moderate and symptoms reappear when the treatment period is discontinued. Therefore, a continuous long-term treatment or prevention with compounds which have less side effects as SSRIs and can be taken over long time periods might by favourable over drug treatment.

There is a need for compounds for the treatment or prevention of mental diseases and/or disorders which do not show the negative side effects of known antidepressants. Many patients are interested in alternative therapies which could minimize the side effects associated with high-dose of drugs and yield additive clinical benefits. Severe depression is a long lasting and recurring disease, which is usually poorly diagnosed. Furthermore many patients suffer from mild or middle severe depression. Thus, there is an increasing interest in the development of compounds as well as pharmaceutical and/or dietary compositions that may be used to treat mental diseases/disorders or to prevent the development of mental diseases/disorders such as depression and disthymia in people at risk, and to stabilize mood.

It is know that imbalanced mood is connected to impaired, i.e., reduced neurotransmission, and that this also leads to increased susceptibility to stress.

Thus, mood is influenced by neurotransmitter biosynthesis, neurotransmitter processing, neurotransmitter storage, neurotransmitter release, neurotransmitter re-uptake and neuro-transmitter receptor binding, especially wherein serotonin is the neurotransmitter. Imbalanced mood may manifest itself in animals including humans as tension, sadness, unhappiness/discontent, irritability and dysphoria, and/or as a disturbance of behaviour, emotions and thinking processes.

Thus, there is also a need for compounds to harmonize or normalize mood and achieve emotional balance to cope with daily life stress and to maintain physical and psychological performance.

Mood disorders and occupational stress also lead to consecutive sleep disorders, (insomnia) delayed sleep onset and low sleep quality, disturbances in cireadian rhythms (so-called bio-rhythm). These conditions are often chronic and can persist over long time. Also, deregulation of circadian rhythms induced by long-distance flights (jet-lag) as well as by shift-working can cause similar symptoms and distress. Therefore, treatment with dietary supplementation to alleviate and prevent symptoms associated with the sleep disorders, as well as to maintain the normal circadian rhythm (an animal or human is used to), and/or to alleviate and prevent symptoms associated with a disturbed circadian rhythm, such as impairment of cognitive function and memory, mental and physical fatigue, dreaminess, is warranted to improve the overall quality of life and benefiting vital energy of a person in need thereof.

### DESCRIPTION OF THE INVENTION

Surprisingly, it has been found that compounds of the general formula I as defined below are effective serotonin and noradrenaline re-uptake inhibitors, and inhibitors of monaminooxidase A and B. Thus they have similar properties as currently commercialized anti-depressants drugs like SSRIs (selective serotonin-reuptake inhibitors), SNRIs (selective noradrenaline-reuptake inhibitors) and tricyclic amines)

The present invention refers especially to the use of the compounds of the general formula I as mood balancing agents and stress relievers, for mental well-being as well as to helping to reduce the risk of mood swings, for helping to retain a positive mood and for supporting cognitive wellness, for normalizing the sleep pattern (also called sleep architecture), shorten the time to sleep onset, in general for helping to promote and maintain a good sleep quality, as well as (dietary) compositions and fortified food/feed/beverages containing them, and their uses. These compounds (especially ligustilide, 3n-butylphtalide, senkyunolide A, 13, C, cnidilide: are preferably used for normalizing and maintaining the circadian rhythm (biorhythm) in humans, for alleviating and/or preventing the symptoms associated with a disturbed circadian rhythm (biorhythm) in humans.

Additionally the compounds of this invention have veternary uses, including: preventing stress in farm animals in mass production lifestock husbandry and/or during transport to slaughter; preventing quality loss of meat of the farm animals during transport to slaughter; for preventing feather picking and cannibalism amongst poultry; and for reduction of stress in pets.

According to the present invention this demand is met with known compounds of the general formula I,

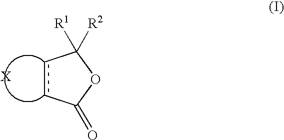

wherein
R¹ is hydrogen or hydroxy;
with the proviso that R² is butyl or butyryl if R¹ is hydroxy, and R² is butyl if R¹ is hydrogen; or
R¹ and R² taken together are 1-propylidene or 1-butylidene optionally substituted by hydroxy, methyl, or 3-(α, β-dimethylacryloyloxy);
the dotted line is an optional bond;
X is an optionally substituted aliphatic C4-residue selected from the group consisting of X1, X2, X3, X4, and X5;

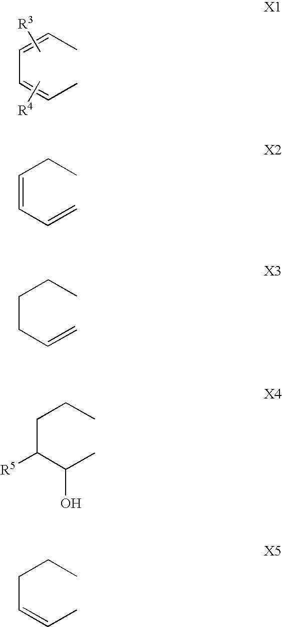

wherein
X is X2, X3 or X5 if the dotted line in formula (I) is absent; and
X is X1, X4 or X5 if the dotted line signifies a bond in formula (I) above;
R³ and R⁴ are, independently from each other, hydrogen or hydroxy; and
R⁵ is hydroxy or butyryl.

Thus, in a compound of formula (I) above X1 can be substituted by one (if one of R³ and R⁴ is hydroxy) or two hydroxy groups (if R³ and R⁴ are both hydroxy).

The compounds of the general formula I as described above act as serotonin re-uptake inhibitors, thus prolonging the time the serotonin is available for neurotransmission. This leads to a mood balancing and stress relieving effect.

The compounds as used for the present invention are selected from the group of phthalide derivatives, which refer to substituted lactones of 2-hydroxymethylbenzoic acid according to IUPAC Rule C-473. This class of compounds is based on 1(3H)-isobenzofuranone C₈H₆O₂.

Preferred compounds used for the purpose of the present invention are selected from the group consisting of (E)-senkyunolide E; senkyunolide C; senkyunolide B; 3-butyl-4,5,6,7-tetrahydro-3,6,7-trihydroxy-1(3H)-isobenzofuranone; 3-butyl-1(3H)-isobenzofuranone; 3-butylphthalide; 3-butylidenephthalide; 3-propylidenephthalide; chuangxinol; ligustilidiol; senkyunolide F; 3-hydroxy-senkyunolide A; angeloylsenkyunolide F; senkyunolide M; 3-hydroxy-8-oxo-senkyunolide A; ligustilide; 6,7-dihydro-(6S,7R)-dihydroxyligustilide; 3a,4-dihydro-3-(3-methylbutylidene)-1(3H)-isobenzofuranone; sedanolide and cnidilide, as well as mixtures thereof. The preferred embodiments are listed in Table 1.

TABLE 1
List of preferred compounds used according to the present invention

| Structure | Name |
|---|---|
| 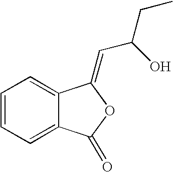 | (E)-Senkyunolide E |
| 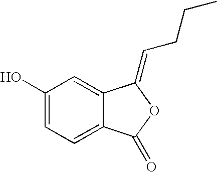 | Senkyunolide C |
| 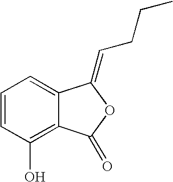 | Senkyunolide B |
| 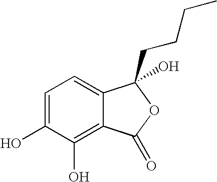 | 3-Buty1-4,5,6,7-tetrahydro-3,6,7- trihydroxy-1(3H)-isobenzofuranone |
| 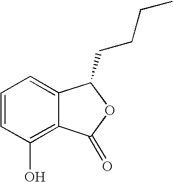 | 3-Butyl-1(3H)-isobenzofuranone |
| 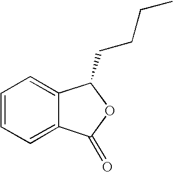 | 3-n-Butylphthalide |
| 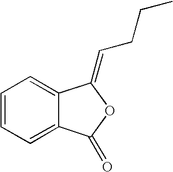 | 3-Butylidenephthalide |
| 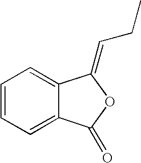 | 3-Propylidenephthalide |
| 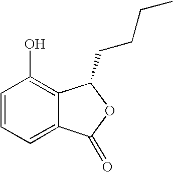 | Chuangxinol |
| 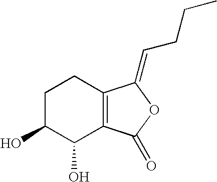 | Ligustilidiol |
| 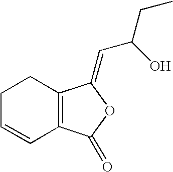 | Senkyunolide F |
| 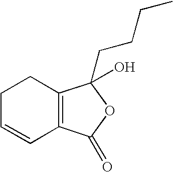 | 3-Hydroxy-senkyonolide A |
| 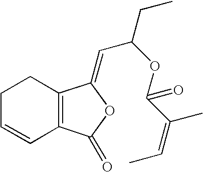 | Angeloylsenkyunolide F |
| 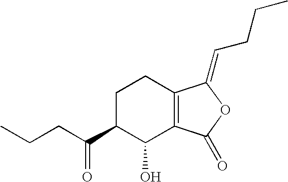 | Senkyunolide M |
| 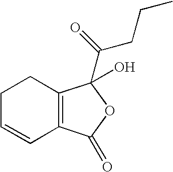 | 3-Hydroxy-8-oxo-senkyunolide A |
| 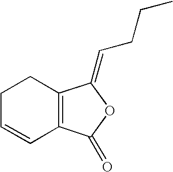 | Ligustilide |
| 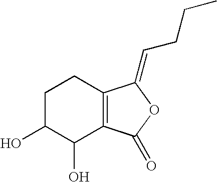 | 6,7-Dihydro-(6S,7R)-dihydroxyligustilide |
| 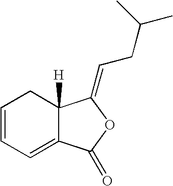 | 3a,4-Dihydro-3-(3-methylbutylidene)- 1(3H)-isobenzofuranone |
| 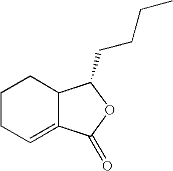 | Sedanolide |
| 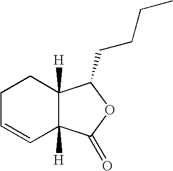 | Cnidilide |

The most preferably used compounds are selected from the group consisting of ligustilide, 3-butylphthalide, 3-butylidenephthalide, 3-propylidenephthalide, sedanolide, senkyunolide A, senkyunolide B and senkyunolide C, as well as mixtures thereof.

Thus, in one aspect the invention relates to a compound of the general formula I with the definitions of X and R¹-R⁵ as given above, preferably to a compound selected from the group consisting of (E)-senkyunolide E; senkyunolide C; senkyunolide B; 3-butyl-4,5,6,7-tetrahydro-3,6,7-trihydroxy-1(3H)-isoberizofuranone; 3-butyl-1(3H)-isobenzofuranone; 3-butylphthalide; 3-butylidenephthalide; 3-propylidenephthalide; chuangxinol; ligustilidiol; senkyunolide F; 3-hydroxy-senkyunolide A; angeloylsenkyunolide F; senkyunolide M; 3-hydroxy-8-oxo-senkyunolide A; ligustilide; 6,7-dihydro-(6S,7R)-dihydroxyligustilide; 3a,4-dihydro-3-(3-methylbutylidene)-1(3H)-isobenzofuranone; sedanolide; and enidilide, as well as to a mixture thereof.

More preferably, the compound is selected from the group consisting of ligustilide, 3-butylphthalide, 3-butylidenphthalide, 3-propylidenephthalide, sedanolide, senkyunolide A, senkyunolide B and senkyunolide C, as well as to a mixture thereof, for use in the treatment of a disorder connected to impaired or reduced neurotransmission or for use in the manufacture of compositions useful for the treatment of a disorder connected to impaired neurotransmission.

Thus, in one aspect the invention relates to the use of a compound of the general formula I to as defined above, preferably use of a compound listed in Table 1, more preferably to the use of ligustilide, 3-butylphthalide, 3-butylidenephthalide, 3-propylidenephthalide, sedanolide, senkyunolide A, senkyunolide B and senkyunolide C, as well as mixtures thereof, as mood balancing agent and/or stress reliever in animals including humans.

The animals are especially humans, pet or companion animals (preferably cats and dogs), farm animals (preferably poultry, cattle, sheep, goat and swine) and animals for the fur industry such as minks, foxes and hares, as well as animals used for aquaculture such as fish like salmon and trout as well as crustaceous like shrimp.

In one preferred embodiment of the present invention the compounds of this invention are used for preventing stress in pet and farm animals, in mass production lifestock husbandry, during transport to slaughter and/or for preventing losses in milk or egg production, and quality loss of meat of said farm animals during transport to slaughter.

In another preferred embodiment of the present invention the compounds of this invention are used for preventing feather picking and cannibalism amongst poultry and/or for preventing losses of meat quality and egg production accompanied by it and in general.

In a further preferred embodiment of the present invention the compounds of this invention are used for maintaining the circadian rhythm in humans, for alleviating and/or for preventing the symptoms associated with a disturbed circadian rhythm in humans. Thus, mood is stabilized and an emotional balance is achieved to cope with daily life stress and to maintain physical and psychological performance. Furthermore, the symptoms associated with a disturbed circadian rhythm such as impairment of cognitive function and memory, and mental and physical fatigue are alleviated and/or prevented so that the overall quality of life is improved and the persons to whom such compounds are administered benefit from maintaining vital energy. Also, deregulation of circadian rhythms induced by long-term flights (jet-lag) as well as by shift-working and the symptoms associated with it are alleviated and/or prevented.

Thus, imbalanced mood which may manifest as tension, sadness, unhappiness/discontent and irritability, dysphoria, and/or as a disturbance of behaviour, emotions and thinking processes, is prevented by administering the compounds of the general formula I as defined above to animals including humans.

In another aspect, the invention relates to the use of a compound of the general formula I as defined above for the treatment of a disorder connected to impaired or reduced neurotransmission or for the manufacture of a composition for the treatment of a disorder connected to impaired neurotransmission, particularly for the manufacture of an antidepressant, a reducer of obsessive-compulsive behaviour, a relaxant, a sleep improver and/or a insomnia alleviator, as well as for use as mood balancing agent, as mood/vitality improver, as stress reliever, as condition improver and/or as reducer of tension, sadness, unhappiness/discontent, irritability and dysphoria.

In still another aspect, the invention relates to compositions, particularly to dietary compositions containing at least one compound of the general formula I as defined above as well as to pharmaceutical compositions containing at least one compound of the general formula I as defined above and a conventional pharmaceutical carrier for use in the treatment of disorders connected to impaired neurotransmission.

Further, the invention relates to a method for the treatment of a disorder connected to impaired neurotransmission in animals including humans, said method comprising (a) preparing a composition comprising a compound of formula I and (b) administering an effective dose of said composition to animals including humans which are in need thereof.

Further, the invention relates to a method balancing the mood and/or relieving the stress in animals including humans, said method comprising administering an effective dose of a compound of the general formula I as defined above and with to animals including humans which are in need thereof.

Animals in the context of the present invention include humans and encompass mammals, fish and birds. Preferred “animals” are humans, pet or companion animals, farm animals, animals for the fur industry and in aquaculture. More preferred “animals” are humans, pets and farm animals.

Examples for pet animals are dogs, cats, birds, aquarium fish, guinea pigs, (jack) rabbits, hares and ferrets.

Examples of farm animals are fish (such as e.g. salmon and trout), aqua culture animals (such as e.g. shrimp), pigs, horses, ruminants (cattle, sheep and goat) and poultry (such as e.g. geese, chicken, broiler, laying hens, quails, ducks, turkeys).

Examples of animals for the fur industry are minks, foxes and hares.

In a preferred embodiment of the present invention a compound of the general formula I as defined above is administered for preventing stress in farm animals, in mass production lifestock husbandry, during transport to slaughter and/or for preventing quality loss of meat of said farm animals during transport to slaughter. Said farm animals are especially poultry (such as e.g. geese, broiler, laying hens, quails, ducks, chicken, turkeys), cattle, sheep, goat and swine.

In another preferred embodiment of the present invention the compound of the general formula I as defined above is administered to pet animals for reduction of stress, tension and aggressiveness and compulsive behavior under stressfully conditions such as separation, change or loss of the owner, during holiday separation and husbandry in so called “animal hotels”, husbandry in animal shelter stations, and other conditions of dense husbandry and breeding.

In another preferred embodiment of the present invention the compound of the general formula I as defined above is administered to poultry (such as e.g. geese, broiler, laying hens, quails, ducks, chicken, turkeys) for preventing feather picking and cannibalism resulting e.g. in losses of meat quality and egg production.

In a further preferred embodiment of the present invention the compound of the general formula I as defined is administered to humans for maintaining the circadian rhythm in humans, for alleviating and/or preventing the symptoms associated with a disturbed circadian rhythm in humans.

The term “compound of the general formula I” also encompasses any material or extract of a plant containing such a compound covered by the general formula I, preferably in an amount of at least 50 weight-%, more preferably in an amount of from 70 to 90 weight-%, most preferably in an amount of at least 90 weight-%, based on the total weight of the plant material or extract. The terms “material of a plant” and “plant material” used in the context of the present invention mean any part of a plant.

The compounds according to formula I as defined above and which are used according to the present invention may be isolated by methods known in the art [see, e.g. Beck J. et al 1995 Natural Products 58(7): 1047-1055] from various plants such as Angelica glauca, Angelica acutiloba, Angelica sinensis, Angelicae dahuricae, Apium graveolens, Ligusticum acutilobum, Ligusticum officinale, Ligusticum sinense, Ligusticum wallichii, Ligusticum chuanxiong, Cnidium officinale, Rhizoma chuanxiong, Pleurospermum hookeri, Trachyspermum roxburghianum, Meum athamanticurn, Lomatium torreyi, Scutellaria baicalensis, Opopanax chironium, Cenolophium denudatum, Coriandrum sativuum, and Silaum silaus. Therefore, any material, extract or oleoresin of these plants or any other plant material or extract containing a specific compound covered by formula I, especially in an amount of at least 50 weight-%, preferably in an amount of from 70 to 90 weight-%, more preferably in an amount of at least 90 weight-%, based on the total weight of the plant material or extract, is also encompassed by the expression “compound of the formula I”.

The compounds used herein may also be of synthetic origin. Thus, “compound of the formula I” means both “natural” (isolated) and “synthetic” (manufactured) compound of the formula I. In this case both cis (Z) and trans (E)-isomers are included.

3-n-Butylphthalide, senkyunolides A, B and C and sedanolide may be obtained by distillation and fractionation of Apium graveolens seed extract, or by supercritical fluid CO2 extraction, and if desired, followed by a distillation. Supercritical fluid extract/distillation is a preferred method for extracting Ligusticum.

Beside the pure compounds of formula I especially preferred are plant materials and plant extracts containing at least 50 weight-%, more preferably from 70 to 90 weight-%, most preferably at least 90 weight-%, of these compounds, based on the total weight of the plant material/extract. Extracts of whole plants or parts thereof, e.g., of leaves, roots or seed are commercially available or can be obtained in accordance with methods well-known in the art using solvents like methanol, ethanol, ethyl acetate, diethyl ether, n-hexane, methylenechloride or, preferably, with supercritical fluids like carbon dioxide (pure or in mixture with other solvents such as alcohols) or dinitrogen oxide. From the extracts single compounds or more or less pure mixtures can be obtained by distillation and fractionation, preferably under reduced pressure. Alternatively, pure compounds of formula I can be synthesized by methods well-known in the art and mixed together in any desired quantities.

In a preferred embodiment of the present invention the compounds of the general formula I are administered in form of a ligusticum distillate, or as a mixture with ligusticum distillate as one component, which contains the following five phthalides:

3-n-butylphthalide,
3-n-butylidenphthalide,
senkyunolide A,
sedanolide and
Z-ligustilide. Preferably this distillate/mixture contains at least 40 weight-%, preferably at least 55%, more preferably at least 65%, most preferably at least 85 weight-% of these 5 phthalides together, based on the total weight of the distillate/mixture. In the distillate/mixture,
preferably the amount of Z-ligustilide is:
in an amount in the range of from at least 30 to 60 weight-%;
more preferably at least 40 weight-%,
even more preferably at least 50 weight-%;
   preferably the amount of senkyunolide A is:
in an amount in the range of from 10-40 weight-%;
preferably at least 10 weight-%,
more preferably at least 20 weight-%, and
even more preferably at least 30 weight %;
   preferably the amount of sedanolide is:
in a range of from about 0.1 to 8 weight %
preferably at least 0.5% weight %
more preferably at least 1.0 weight-%,
even more preferably at least 3 weight-%, and
   preferably the amount of 3-n-butylphthalide is
in a range of from about 0.1 at least 5 weight %,
preferably at least 0.5 weight-%,
more preferably at least 2% weight-%,
even more preferably at least 3 weight-%,
   all amounts based on the total weight of the distillate/mixture.

The dietary compositions according to the present invention may further contain protective hydrocolloids, binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

The term “dietary compositions” comprises any type of nutrition such as (fortified) food/feed and beverages including also clinical nutrition, and also dietary supplements.

Beside a pharmaceutically acceptable carrier and at least one compound of the general formula I with the definitions of X, R¹-R⁵ and the preferred ones as given above, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administration to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules. Examples for other application forms are forms for transdermal, parenteral or injectable administration. The dietary and pharmaceutical compositions may be in the form of controlled (delayed) release formulations.

Examples of food are dairy products such as yoghurts.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, near-water drinks (i.e. water based drinks with a low calorie content), teas and milk based drinks. Liquid food are e.g. soups and dairy products (e.g. muesli drinks).

The compound of the general formula I with the definitions of X, R¹ and R² and the preferences as given above can be used for the manufacture of medicaments for the treatment of a disorder connected to impaired or reduced neurotransmission.

In the context of this invention “treatment” also encompasses co-treatment as well as prevention. “Prevention” can be the prevention of the first occurrence (primary prevention) or the prevention of a reoccurence (secondary prevention).

Thus, the present invention is also directed to a method for the prevention of a disorder connected to impaired neurotransmission in animals including humans, said method comprising administering an effective dose of a compound of the general formula I with the definitions of X, R¹ and R² and the preferences as given above to animals including humans which are in need thereof. In this regard an effective dose of a compound of the general formula I with the definitions of X, R¹ and R² and the preferences as given above may especially be used for maintaining the mental well-being, for maintaining a balanced cognitive function, for helping to reduce the risk of mood swings, for helping to retain a positive mood and for supporting cognitive wellness, for normalizing the sleep pattern (also called sleep architecture), shorten the time to sleep onset, in general for helping to promote and maintain a good sleep quality, normalize the circadian rhythm (biorhythm) of one person, meaning restoring the biorhythm of a person to his usual, healthy one.

In the context of this invention the term “disorder” also encompasses diseases.

Pharmaceutical and dietary compositions for the treatment of disorders connected to impaired neurotransmission encompass their use as antidepressants, mood/vitality improvers, stress relievers, condition improvers, anxiety reducers and obsessive-compulsive behaviour reducers, relaxants, sleep improvers and/or insomnia alleviators, normalizer of circadian rhythm (biorhythm). They all may improve, enhance and support the physiological neurotransmission, especially in the central nervous system, and therefore may alleviate mental malfunction.

Antidepressants are medicaments for treating mental, behavioural and emotional/affective, neurotic, neurodegenerative, eating and stress related disorders such as e.g. unipolar depression, bipolar depression, acute depression, chronic depression, subchronic depression, dysthymia, postpartum depression, premenstrual dysphoria/syndrom (PMS), climacteric depressive symptoms, aggression, attention deficit disorders (ADS), social anxiety disorders, seasonal affective disorders, anxiety (disorders) such as generalized anxiety disorder (GAD), fibromyalgia syndrome, chronic fatigue, sleep disorders (insomnia), post-traumatic stress disorders, panic disorders, obsessive compulsive disorders, restless leg syndrome, nervousness, migraine/primary headaches and pain in general, emesis, bulimia, anorexia nervosa, binge eating disorder, gastrointestinal disorders, burn out syndrome, irritability and tiredness.

Antidepressants can also be used for (the manufacture of compositions for) primary and secondary prevention and/or the treatment of neurocognitive impairment. Furthermore they are also effective in the treatment of depressive symptoms or other symptoms related to disturbed neurotransmission occurring as comorbidity in chronic diseases such as cardiovascular diseases, strokes, cancer, Alzheimer disease, Parkinson disease, and others.

The compound of the general formula I with the definitions of X, R¹ and R² and the preferences as given above as well as (mixtures of) plant materials and plant extracts (essentially) containing them (especially in an amount of at least 30 weight-%, preferably in an amount of at least 50 weight-%, more preferably in an amount of from 70 to 90 weight-%, most preferably in an amount of at least 90 weight-%, based on the total weight of the plant material or extract) and dietary/pharmaceutical compositions containing them are thus suitable for the treatment of animals including humans.

Especially companion (pet) animals and livestock (farm) animals can be in conditions in need of enhanced or improved neurotransmission. Such conditions e.g. occur after capture or transport or with housing, when the animals develop analogous disorders and are distressed or aggressive, or display stereotypic behaviour, or anxiety and obsessive-compulsive behaviour.

Thus, the compounds of the general formula I with the definitions of X, R¹ and R² and the preferences as given above can be used in general as antidepressants for animals including humans, preferably for humans, companion (pet) animals and farm animals.

In a further embodiment of the present invention the compounds of the general formula I with the definitions of X, R¹ and R² and the preferences as given above find use as mood improver in general as well as for the manufacture of compositions for such use (plant materials/extracts; dietary/pharmaceutical compositions). “Mood improver” or “emotional wellness booster” or “vitality improver” means that the mood of a person treated with it is enhanced, that the self esteem is increased and/or that negative thoughts and/or negative tension are/is reduced. It also means the emotions are balanced and/or that the general, especially the mental, well being and vitality is improved or maintained, as well as that the risk of mood swings is (helped to be) reduced and that the positive mood is (helped to be) retained.

The compounds of the general formula I with the definitions of X, R¹ and R² and the preferences as given above can also be used in general as anxiety reducer and/or obsessive-compulsive behaviour reducer for animals including humans, preferably for humans, pet animals and farm animals.

Anxiety reducer means that chronic tension and anxious worrying and tension are alleviated and relieved. Hypervigilance syndrome, including restlessness, muscle tension, and sleep problems are relieved. Social and other phobias are resolved. In general, the social environment is experienced less threatening. The person is emotionally relaxed, experiences comfort and enjoys company and contact to other people.

“Relaxant” or “sleep improver” or “insomnia alleviator” means improving sleep onset (shortening time to enter sleep, lag time to sleep) and helping a person to easily enter sleep, normalize sleep patterns, or to maintain an undisrupted sleep over the night. It also means to correct circadian rhythm associated sleep disturbances due to jet-lag or shift work, and to prevent and abolish the symptoms associated with sleeplessness, i.e. impairment of cognitive function and memory, mental and physical fatigue, dreaminess, and improve overall quality of life and vital energy.

Moreover, the compounds of the general formula I with the definitions of X, R¹ and R² and the preferences as given above as well as compositions comprising an effective dose of them are useful for the treatment, prevention and the alleviation of stress related symptoms, for the treatment, prevention and alleviation of symptoms related to working overload, exhaustion and/or burn out, for the increase of the resistance or tolerance to stress and/or to favor and facilitate the relaxation in normal healthy individuals i.e. such compositions have an effect as “stress reliever”.

Furthermore, the compounds of the general formula I with the definitions of X, R¹ and R² and the preferences as given above as well as compositions comprising an effective dose of them are useful for the treatment, prevention and alleviation of anxiety and obsessive-compulsive behaviour in humans and animals.

A further embodiment of the present invention relates to the use of compounds of the general formula I with the definitions of X, R¹ and R² and the preferences as given above and to the use of compositions containing them (plant materials/extracts; dietary/pharmaceutical compositions) as “condition improver”, i.e. as means to reduce irritability and tiredness, to reduce or prevent or alleviate physical and mental fatigue, and to increase energy in more general terms, especially to increase the brain energy production, in diseased or normal healthy individuals. Moreover for cognition improvement in general, and especially for maintenance or improvement of attention and concentration, of the memory and of the capacity for remembering, of the learning ability, of the language processing, of problem solving and of intellectual functioning; for improvement of the short-term memory; for increasing the mental alertness and sharpness, capability to focus; for enhancing the mental vigilance; for reducing the mental fatigue; for supporting cognitive wellness, for maintaining balanced cognitive function, for the regulation of hunger and satiety as well as for the regulation of motor activity.

The present invention not only refers to compounds of the general formula I with the definitions of X, R¹ and R² and the preferences as given above and their compositions (i.e. (mixture(s) of) plant extracts (essentially) containing them; dietary/pharmaceutical compositions containing them) for use as medicaments, especially for the treatment of disorders connected to impaired neurotransmission, but also for the methods for the treatment of such disorders themselves, as already mentioned above.

In an especially preferred embodiment of such method pet animals or farm animals whose disorders are associated with housing, capture or transport are treated and which may appear in form of anxiety or obsessive-compulsive behaviour.

For humans a suitable daily dosage of a compound of the general formula I with the definitions of X, R¹ and R² and the preferences as given above, for the purposes of the present invention may be within the range from 0.001 mg per kg body weight to about 20 mg per kg body weight per day. More preferred is a daily dosage of about 0.01 to about 10 mg per kg body weight, and especially preferred is a daily dosage of about 0.05 to 5.0 mg per kg body weight. The amount of a plant material or plant extract containing such compound of the general formula I can be calculated accordingly.

In solid dosage unit preparations for humans, the compound of the general formula I with the definitions of X, R¹ and R² and the preferences as given above, is suitably present in an amount from about 0.1 mg to about 1000 mg, preferably from about 1 mg to about 500 mg per dosage unit.

In dietary compositions, especially in food and beverages for humans, the compound of the general formula I with the definitions of X, R¹ and R² and the preferences as given above, is suitably present in an amount of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage.

In food and drinks in a preferred embodiment of the invention the amount of the compound of the general formula I with the definitions of X, R¹ and R² is 10 to 30 mg per serving, i.e. 120 mg per kg food or drink.

For animals excluding humans a suitable daily dosage of the compound of the general formula I with the definitions of X, R¹ and R² and the preferences as given above, for the purposes of the present invention may be within the range from 0.001 mg per kg body weight to about 1000 mg per kg body weight per day, More preferred is a daily dosage of about 0.1 mg to about 500 mg per kg body weight, and especially preferred is a daily dosage of about 1 mg to 100 mg per kg body weight.

For companion (pet) animals the same daily dosages as for humans are suitable.

The invention is illustrated further by the following examples.

### EXAMPLES

The 3-n-butylphthalide used in the experiments described below was obtained from Advanced Synthesis, PO Box 437920, San Ysidro, Calif. 92173. The senkyunolides and cnidilide were obtained from AnalytiCon Discovery GmbH, Hermannswerder Haus 17, 14473 Potsdam, Germany, and the ligustilide was synthesized by chemists of DSM Nutritional Products Ltd., Kaiseraugst, Switzerland. All compounds were >95% pure.

### Example 1

### Serotonin Uptake Inhibition and Labeled Citalopram Displacement by Compounds of the Formula I

HEK-293 cells stably expressing the human serotonin re-uptake transporter (hSERT) were obtained from R. Blakely, Vanderbilt University, USA. The cells were routinely grown in Dulbeco's Modified Eagles Medium, purchased from Bioconcept, Allschwil, Switzerland containing 10% fetal calf serum, penicillin, streptomycin, L-glutamine and the antibiotic G418 and passaged by trypsinisation. One day prior to the assay cells were seeded in the above mentioned medium. Immediately prior to the assay the medium was replaced by Krebs Ringers bicarbonate buffer, purchased from Sigma Chemicals Ltd., supplemented with 35 μM pargyline, 2.2 mM CaCl₂, 1 mM ascorbic acid and 5 mM N-2-hydroxyethylpiperazine-N′-2-ethanesulfonic acid (buffer called “Hepes”). Serotonin uptake into the cells was determined by addition of radio-labeled (3H) serotonin (Amersham Biosciences GE Healthcare, Slough, UK) to a concentration of 20 nM, and incubation for 30 minutes at room temperature. Following removal of unincorporated label by gentle washing three times with the above buffer, incorporated serotonin was quantified by liquid scintillation counting.

The effect of the compounds of formula I on the serotonin uptake was determined by its inclusion in the assay at a range of concentrations between 0.03 and 100 μM for 10 minutes prior to and during the addition of (3H) serotonin/citalopram. Serotonin uptake via the transporter was inhibited by ligustilide, 3-butylphthalide, senkyunolide A, senkyunolide C, senkyunolide B, and cnidilide, respectively, in a dose dependent manner, with IC₅₀ values as shown in Table 2.

TABLE 2
Inhibition of serotonin uptake into transfected
HEK-293 cells by compounds of formula I.

| | IC₅₀ for tritiated |
|---|---|
| Substance | serotonin uptake. |
| Ligustilide | 20.4 +/− 5.8 μM (n = 4) |
| 3-n-butylphthalide | 35.2 +/− 6.7 μM (n = 3) |
| Senkyunolide A | 32.5 +/− 9.1 μM (n = 2) |
| Senkyunolide C | 31.4 +/− 10.9 μM (n = 2) |
| Senkyunolide B | 75.0 +/− 11.0 μM (n = 2) |
| Cnidilide | 40.5 +/− 15.0 μM (n = 3) |
| Celery extract | 99.2 +/− 5.8 μg/ml (n = 2) |
| Wild celery extract | 29.9 +/− 8.3 μg/ml (n = 2) |

Since the experiments were carried out twice or more (n) the average value +/− the standard error in the mean (s.e.m.) is shown.

### Example 2

### Monoaminooxidase Inhibition by Compounds of the Formula I

The organic amines p-tyramine or benzylamine were used as substrates for the Monoamine oxidase A (MAO-A) and B (MAO-B) enzymes respectively. The H₂O₂ produced by this reaction was quantified by reaction with vanillic acid, catalysed by horse radish peroxidase (HRP).

The reactions were carried out in polystyrene microtitre plates. The MAO enzymes (final concentration 2 U/ml) were mixed with either p-tyramine (Sigma, final concentration 0.5 mM) or benzylamine (Sigma, final concentration 0.5 mM) as appropriate and the chromogenic solution (containing vanillic acid (Fluka), 4-aminoantipyrine (Fluka) and horse radish peroxidase (Sigma), final concentrations 0.25 mM, 0.125 mM and 1 U/ml respectively) in 0.2 M potassium phosphate buffer pH 7.6. The reactions were followed in a microtitre plate absorbance reader eg Spectramax M5 (Molecular Devices Corporation). Absorbance readings at 495 nm were taken every 15 seconds for 40 minutes and the initial reaction velocities calculated by linear regression using SOFTmaxPro (Molecular Devices Corporation).

The effect of ligustilide, 3-butylphthalide, senkyunolide C and cnidilide on the monoamine oxidase enzymes was determined by their inclusion in the assay at a range of concentrations between 0.03 and 100 μM for 10 minutes prior to and during the incubation with substrate. To determine the effect of the compounds on the HRP catalyzed portion of the reaction, the MAO enzyme was replaced by H₂O₂ (Molecular Probes, final concentration 0.2 mM). The reactions containing MAO-A and MAO-B were both inhibited by tricyclic diterpenes and their derivatives according to formula I in a dose dependent manner, whilst the control reaction, was unaffected. The measured IC50 values for inhibition of monoamine oxidase activity by ligustilide, 3-butylphthalide, senkyunolide C and enidilide, respectively, are shown in Table 3.

TABLE 3
Inhibition of MAO-A and MAO-B by compounds of the formula I

| | IC50 [μM] for Inhibition | IC50 [μM] for Inhibition |
|---|---|---|
| Substance | of MAO-A | of MAO-B |
| Ligustilide | 15.7 +/− 0.2 s.e.m. n = 2 | 24.9 +/− 3.7 s.e.m. n = 2 |
| 3-n- | 8.1 +/− 1.2 s.e.m. n = 2 | 56.1 +/− 10.2 s.e.m. n = 2 |
| butylphthalide | | |
| Senkyunolide | 2.72 +/− 0.16 s.e.m, n = 2 | 2.14 +/− 0.24 s.e.m. n = 2 |
| C | | |
| Cnidilide | >100 uM n = 2 | 108.8 +/− 17.7 s.e.m. n = 2 |

### Example 3

### Effect of Compounds of the Formula I in the Primary Observation (Irwin) Test in the Mouse

The method, which detects the first toxic dose, the active dose-range and the principal effects of a test substance on behavior and physiological function, follows that described by Irwin 1968 Psychopharmaco, 13:222-257,

Mice were administered the test substance (3-n-butylphthalide, ligustilide) and were observed in simultaneous comparison with a control group given vehicle (non-blind conditions). 3 treated groups were compared with the same control at any one time. All animals within a treatment group were observed simultaneously.

Behavioral modifications, physiological and neurotoxicity symptoms, pupil diameter and rectal temperature were recorded according to a standardized observation grid derived from that of Irwin, supra. The grid contains the following items: lethality*, convulsions*, tremor*, Straub*, sedation, excitation, abnormal gait* (rolling, tip-toe), jumps*, motor incoordination*, writhes*, piloerection*, stereotypies* (sniffing, chewing, head movements), head twitches*, scratching*, respiration*, aggressiveness*, fear, reactivity to touch, muscle tone, loss of righting reflex, ptosis, exopthalmos, loss of grasping, akinesia, catalepsy, loss of traction, loss of corneal reflex, analgesia, defecation, salivation, lacrimation, pupil diameter (Unit=1/45 mm) and rectal temperature.

Observations were performed 15, 30, 60, 120 and 180 minutes after administration of the test substance and also 24 hours later. The symptoms marked (*) were observed continuously from 0 to 15 minutes after administration. 5 mice were studied per group.

3-Butylphthalide (98.5% purity) was solubilized in 3% DMSO, 3% Tween 80 in saline (0.9% w/v NaCl) and injected into mice intraperitoneally.

### Results

3-n-butylphthalide

At 100 mg/kg body weight, 3-n-butylphthalide showed slight sedative effects (in 5/5 mice after 15 and 30 minutes, and 2/5 at 60 minutes) and reduced muscle tone (in 5/5 mice after 15 and 30 minutes, and 3/5 mice after 60 minutes).

At 200 mg/kg body weight it showed moderate sedative effects in 5/5 mice after 60 and 120 minutes), reduced fear and reduced muscle tone after 60 minutes.

At 300 mg/kg body weight, it showed marked sedation in 4/4 mice after 15 minutes and 30 minutes, moderate sedation in 4/4 mice at 60 minutes and 120 minutes, and slight sedation in 4/4 mice at 180 minutes.

3-Butylphthalide at 300 mg/kg also reduced fear and muscle tone at 120 minutes.

Overall, 3-butylphthalide showed a dose dependent sedative, fear reducing and muscle relaxant effect.

Ligustilide (98.9% purity) at 30 mg/kg induced slight sedation in 3 mice at 30 minutes.

At 100 mg/kg it induced slight sedation in all 5 mice from 15 to 30 minutes and in 3 mice at 60 minutes; it decreased muscle tone in 2 mice at 30 minutes.

At 200 mg/kg sedation was slight-to-moderate in all 5 mice from 15 to 120 minutes and slight in 2 mice at 180 minutes; it decreased muscle tone in all 5 mice at 15 minutes, in 4 mice from 30 to 60 minutes, in 2 mice at 120 minutes and in 1 mouse at 180 minutes. At 300 mg/kg sedation was moderate in all 5 mice from 15 to 60 minutes and slight from 120 to 180 minutes; it decreased muscle tone in all 5 mice from 15 to 30 minutes, in 4 mice from 60 to 120 minutes and in 3 mice at 180 minutes.

Overall, 3-n-butylphthalide and ligustilide showed a dose dependent sedative and muscle relaxant effect.

### Example 4

### Porsolt's Swim Test with 3-n-Butylphthalide and Ligustilide

The “Behavioural Despair Test” or “Porsolt's Forced Swim Test” is a validated animal model for depression (see T, Nagatsu 2004 Neuro Toxicology, 25:11-20, and Porsolt et al. 1977, Arch. Int. Pharmacodyn., 229: 327-336). It responds to enhancement of the trans-mission of several neurotransmitters including serotonine, dopamine and noradrenaline.

The test, which detects antidepressant activity, was carried out as described by Porsolt et al supra. Mice which are forced to swim in a situation from which they cannot escape rapidly become immobile. Antidepressants decrease the duration of immobility.

Mice were individually placed in a cylinder (Height=24 cm, Diameter=13 cm) containing 10 cm water (22° C.) from which they could not escape. The mice were placed in the water for 6 minutes and the duration of immobility during the last 4 minutes was measured.

10 mice were studied per each of the four groups. The test was performed blind, i.e. the person carrying out the experiment was different from the person injecting the mice and didn't thus know to which of the four groups each mouse belonged.

3-n-butylphthalide (98.5% purity) was evaluated at 3 doses each: at 10, 30 and 80 mg/kg body weight, administered intraperitoneally 30 minutes before the test, and compared with a control group. The thus administered 3-n-butylphthalide was dissolved in a saline solution containing 3 weight-% DMSO and 3 weight-% Tween® 80 (so called “vehicle”), To the control group the vehicle consisting of the saline solution containing 3 weight-% DMSO and 3 weight-% Tween® 80 was administered intraperitoneally.

Results are shown in Table 4.

TABLE 4
Reduction of “immobility time” with 3-n-butylphthalide.

| | Concentration of 3-n- | Duration of immobility |
|---|---|---|
| | butylphthalide | [seconds] ± Standard |
| Group | [mg/kg body weight] | Error of Mean |
| Group 1 | 0 (control group) | 157.4 ± 13.2 |
| Group 2 | 10 | 132.4 ± 9.3 |
| Group 3 | 30 | 128.0 ± 15.3 |
| Group 4 | 80 | 129.0 ± 15.4 |

3-n-butylphthalide between 10 and 80 mg/kg showed a tendency to reduce the immobility time by approximately 18%.

TABLE 5
Reduction of “immobility time” with ligustilide

| | Concentration of | Duration of immobility |
|---|---|---|
| | ligustilide | [seconds] ± Standard |
| Group | [mg/kg body weight] | Error of Mean |
| Group 1 | 0 (control group) | 158.7 ± 10.1 |
| Group 2 | 10 | 128.9 ± 15.1 |
| Group 3 | 30 | 164.2 ± 15.5 |
| Group 4 | 80 | 147.1.0 ± 12.1 |

Ligustilide at 10 mg/kg showed a tendency to reduce the immobility time by approx. 18%, but not at the higher doses.

### Example 5

### Elevated Pluz Maze Test with 3-n-butylphthalide

The method, which detects anxiolytic activity, follows that described by Handley et al, 1984 (Naunyn-Schm Arch Pharm 387: 1-5. Rodents avoid open spaces (the open arms of an elevated plus-maze). Anxiolytics increase exploratory activity in the open anus.

The maze consists of 4 arms of equal length and width (14×5 cm) arranged in the form of a plus sign (+). Two opposite arms are enclosed by 12 cm high walls (closed arms). The 2 other arms have no walls (open arms). The maze is raised 56 cm above the floor. A mouse is placed in the centre of the plus-maze and left to explore for 5 minutes. The number of entries into the open and closed arms and the time spent in the open arms is recorded. 10 mice were studied per group. The test was performed blind.

3-n-Butylphthalide was tested at doses of 10, 30, and 80 mg/kg body weight.

The results are shown in Table 6.

TABLE 6

| TREATMENT | OPEN ARM | | OPEN ARM | |
|---|---|---|---|---|
| (mg/kg) | Number of entries | | Time spent (s) | |
| p.o. −24 h, 5 h | | % | | % |
| and 1 h before test | Mean ± sem | change | Mean ± sem | change |
| Vehicle | 2.4 ± 0.5 | — | 18.2 ± 4.9 | — |
| 3-n-Butylphthalide (10) | 3.5 ± 0.7 | +46% | 24.3 ± 4.6 | +34% |
| 3-n-Butylphthalide (30) | 3.1 ± 0.5 | +29% | 23.0 ± 3.8 | +26% |
| 3-n-Butylphthalide (80) | 3.0 ± 0.8 | +25% | 26.4 ± 5.3 | +45% |

3-n-butylphthalide showed a trend to moderately increase the number of entries and the time spent in the open arm. It had no effect on the entries into the closed arm. 3-n-butylphthalide thus has a moderate anxiolytic activity.

### Example 6

### Marble Burying Test as Test for Anxiety Like or Obsessive Compulsive Behaviour

A celery seed oil extract rich in phthalides was tested in the marble burying test. A crude extract from celery seed oil (Apium graveolens) obtained by supercritical CO₂ fluid extraction of the seed was bought from Guanzhou Honsea Sunshine Bio Science & Technology, Guangzhou, 510620, Peopole's Republic of China. This extract (66.15 g) was distilled for about 2 hours in a ca. 30 cm Kugelrohr at a temperature of 125° C., under a pressure of 0.02 mbar. The distillate contained 18.12% butylphthalide, 0.87% butylidenphthalide, 35.37% senkyanolide, 20.99% sedanolide, 2.23% C16-fatty acid and 3.14% oleic acid, based on the total weight of the celery seed oil extract.

“Defensive burying” behaviour was demonstrated by rats burying noxious objects, such as drinking spouts filled with a unpleasant-tasting liquid (Wilkie et al., 1979 J Exp Anal Behay. 31: 299-306) or shock prods (Pinel et at 1978, J Comp and Physiol Psych 92: 708-712.

Marble burying behaviour by mice is reported to be sensitive to a range of minor (e.g. diazepam) and major (e.g. haloperidol) tranquilisers (Broekkamp et al., 1986 Eur J Pharmacol. 126:223-229), in addition to SSRIs (e.g. fluvoxamine, fluoxetine, citalopram), tricyclic antidepressants (e.g. imipramine, desipramine) and selective noradrenaline uptake inhibitors (e.g. reboxetine), at doses which do not induce sedation. The model may reflect either anxiety-like- or obsessive-compulsive-behaviour (see De Boer et al, 2003 Eur J Pharmacol 463:145-161.

The marble burying test was performed in Type II cages (16×22 cm), which were filled with normal rodent bedding material, up to a depth of 4 cm, and the bedding was flattened gently by hand to ensure an even surface. Twelve marbles were placed, in a 3×4 configuration, evenly spaced, on the surface of the bedding.

At the start of the test, mice were individually placed into the test cages and left to explore for 30 min. At the end of the test, the mice were removed from the cages and the number of marbles which have been buried (i.e. at least ⅔ covered with bedding) were counted. Fresh cages and bedding are prepared prior to testing subsequent animals.

Celery seed oil extract was evaluated after sub-chronic oral administration at 50, 100, 200, 300 mg/kg with fluoxetine at 10 mg/kg as reference substance and corn oil as vehicle. The celery seed oil extract fluoxetine and vehicle were administered by oral gavage 24, −5, −1 h before the test.

After each dose, mice were returned to their homecages until the next dose or beginning of the test.

### Results:

TABLE 7
Effects of celery seed oil extract (Apium graveolens)
and fluoxetine in the marble burying test in mice

| TREATMENT (mg/kg) | MARBLE BURYING TEST |
|---|---|
| p.o., −24, −5, −1 h | % MARBLES BURIED |
| Apium graveolens (50) | −16% |
| Apium graveolens (100) | −4% |
| Apium graveolens (200) | −45% |
| Apium graveolens (300) | −37% |
| Fluoxetine (10) | −78% |

Celery seed oil extract showed a tendency to decrease the number of marbles covered as compared with the vehicle control. The effect was nearly half of that of the selective serotonin reuptake inhibitor fluoxetine.

### Example 7

### Dopamine Uptake Inhibition by Compounds of the Formula I

The actions of several neurotransmitters, including dopamine, are regulated through their to rapid uptake and clearance from synaptic junctions by plasma membrane transport proteins. The dopamine transporter in central dopaminergic neurones is responsible for the recovery of up to 90% of released transmitter. The monoamine transporters are high affinity targets for a number of psychoactive agents such as cocaine, amphetamine, and antidepressants. These agents, by blocking transporters and consequently preventing neuronal uptake, elevate levels of extracellular neurotransmitter concentrations in both the central and peripheral nervous system, contributing to their behavioral and autonomic effects.

CHO-Ki/hDAT cells expressing the human dopamine transporter (hDAT) were plated before the assay. Cells (2×10⁵/ml) were incubated with the compound of the formula I (3-n-butylphthalide) and/or vehicle in modified Tris-HEPES buffer pH 7.1 at 25° C. for 20 minutes before addition of 50 nM [³H]Dopamine for 10 minutes. Specific signal was determined in the presence of 10 μM nomifensine. Cells were then solubilized with 1% SOS lysis buffer. Reduction of [³H]Dopamine uptake by 50 percent or more 50%) relative to vehicle controls indicates significant inhibitory activity. Compounds were screened at 10, 1, 0.1, 0.01 and 0.001 μM. These same concentrations were concurrently applied to a separate group of untreated cells and evaluated for possible compound-induced cytotoxicity only if significant inhibition of uptake was observed

| Compound | IC₅₀ |
|---|---|
| *Nomifensine | 11 nM |
| 3-n-Butylphthalide | 22.0 μM |

*Indicates standard reference agent used.

### REFERENCES

Giros, 1992 Mol. Pharmacol. 42:383-390, 1992.
Pristupa, et al 1994. Mol. Pharmacol. 45: 125-135.

### Example 8

### Norepinephrine Uptake Inhibition by Compounds of the Formula I

The actions of several neurotransmitters, including norepinephrine, are regulated through their rapid uptake and clearance from synaptic junctions by plasma membrane transport proteins. The norepinephrine transporter in central adrenergic neurones is responsible for the recovery of up to 90% of released transmitter. The monoamine transporters are high affinity targets for a number of psychoactive agents such as cocaine, amphetamine, and antidepressants. These agents, by blocking transporters and consequently preventing neuronal uptake, elevate levels of extracellular neurotransmitter concentrations in both the central and peripheral nervous system, contributing to their behavioral and autonomic effects.

Human recombinant norepinephrine transporter stably expressed dog kidney MDCK cells are plated one day before the assay. The cells (2×10⁵/ml) were preincubated with the compound of the formula I (3-n-butylphthalide) and/or vehicle in modified Tri-HEPES buffer pH 7.1 at 25° C. for 20 minutes, then 25 nM [³H]Norepinephrine was added for 10 minutes incubation. Cells in the well were then rinsed twice, solubilized with 1% SDS lysis buffer and the lysate was counted to determine [³H]Norepinephrine uptake. Specific signal was determined in the presence of 10 μM desipramine. Reduction of [³H]Norepinephrine uptake by 50 percent or more (≧50%) relative to vehicle controls indicates significant inhibitory activity. Compounds were screened at 10, 1, 0.1, 0.01 and 0.001 μM. These same concentrations were concurrently applied to a separate group of untreated cells and evaluated for possible compound-induced cytotoxicity only if significant inhibition of uptake was observed.

### Reference Data:

| Inhibitor | IC₅₀ |
|---|---|
| *Desipramine | 1.9 nM |
| 3-n-Butylphthalide | 23.7 μM |

*Indicates standard reference agent used.
^{♦}The experiment was carried out twice

### REFERENCE

Galh et al 1995. J. Exp. Biol. 198:2197-2212.

### Example 9

### Preparation of a Soft Gelatin Capsule

A soft gelatin capsule (500 mg) may be prepared comprising the following ingredients:

| Ingredient | Amount per Capsule |
|---|---|
| Celery seed oil extract | 500 mg |
| Lecithin | 50 mg |
| Soy bean oil | 250 mg |

Two capsules per day for 3 months may be administered to a human adult for the treatment of mild chronic dysthymia.

### Example 10

### Preparation of a Soft Gelatin Capsule

A soft gelatin capsule (600 mg) may be prepared comprising the following ingredients:

| Ingredient | Amount per Capsule |
|---|---|
| Celery seed oil extract | 200 mg |
| Evening primrose oil | 300 mg |
| Vitamin B₆ | 100 mg |

One capsule per day preferably at the second half of the menstrual cycle may be taken for 14 days for the treatment of premenstrual syndrome and premenstrual dysphoric disorder.

### Example 11

### Preparation of a Tablet

A 400 mg-tablet may be prepared comprising the following ingredients:

| Ingredient | Amount per tablet |
|---|---|
| 3-N-butylphthalide | 100 mg |
| Passion flower standardized extract | 150 mg |
| Green Tea Extract, e.g. TEAVIGO ® | 150 mg |
| from DSM Nutritional Products, | |
| Kaiseraugst, Switzerland | |

For general well being, energizing and stress alleviation, one tablet may be taken twice daily for 3 months.

### Example 12

### Preparation of an Instant Flavoured Soft Drink

| Ingredient | Amount [g] |
|---|---|
| 3-N-butylphthalide | 0.9 |
| Sucrose, fine powder | 922.7 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, | 0.4 |
| Kaiseraugst, Switzerland | |
| Total amount | 1000 |

All ingredients are blended and sieved through a 500 μm sieve. The resulting powder is put in an appropriate container and mixed on a turbular blender for at least 20 minutes. For preparing the drink, 125 g of the obtained mixed powder are taken and filled up with water to one liter of beverage.

The ready-to-drink soft drink contains ca. 30 mg of 3-n-butylphthalide per serving (250 ml).

As a strengthener and for general well being 2 servings per day (240 ml) may be drunk.

### Example 13

### Preparation of a Fortified Non Baked Cereal Bar

| Ingredient | Amount [g] |
|---|---|
| 3-N-butylphthalide | 0.95 |
| Sugar | 114.55 |
| Water | 54.0 |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.5 |
| Hardenend palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skim milk powder | 45.0 |
| Apple flavour 74863-33 | 2.0 |
| Citric acid | 5.0 |
| Total amount | 1000 |

3-n-Butylphthalide is premixed with skim milk powder and placed in a planetary bowl mixer. Cornflakes and rice crispies are added and the total is mixed gently. Then the dried and cut apples are added. In a first cooking pot sugar, water and salt are mixed in the amounts given above (solution 1). In a second cooking pot glucose, invert and sorbitol syrup are mixed in the amounts given above (solution 2). A mixture of baking fat, palm-kernel fat, lecithin and emulsifier is the fat phase. Solution 1 is heated to 110° C. Solution 2 is heated to 113° C. and then cooled in a cold water bath. Afterwards solution 1 and 2 are combined. The fat phase is melted at 75° C. in a water bath. The fat phase is added to the combined mixture of solution 1 and 2. Apple flavour and citric acid are added to the liquid sugar-fat mix. The liquid mass is added to the dry ingredients and mixed well in the planetary bowl mixer. The mass is put on a marble plate and rolled to the desired thickness. The mass is cooled down to room temperature and cut into pieces. The non baked cereal bar contains ca. 25 mg oregano extract per serving (30 g). For general well-being and energizing 1-2 cereal bars may be eaten per day.

### Example 14

### Dry Dog Feed Comprising 3-n-Butylphthalide or Ligustilide for Relieving Stress and Revitalizing the Dog

Commercial basal diet for dogs (e.g. Mera Dog “Brocken”, MERA-Tiernahrung GmbH, Marienstraβe 80-84, D-47625 Kevelaer-Wetten, Germany) is sprayed with a solution of 3-n-butylphthalide or ligustilide in an amount sufficient to administer to a subject a daily dose of 50 mg of 3-n-butylphthalide or ligustilide per kg body weight. The food composition is dried to contain dry matter of about 90% by weight. For an average dog of 10 kg body weight to consume approx. 200 g dry feed per day, the dog food contains approx. 2500 mg 3-n-butylphthalide or ligustilide per kg food. For heavier dogs, the feed mix is prepared accordingly.

### Example 15

### Wet Cat Food Comprising 3-n-Butylphthalide or Ligustilide

Commercial basal diet for cats (e.g. Happy Cat “Adult”, Tierfeinnahrung, Südliche Hauptstraβe 38, D-86517 Wehringen, Germany) is mixed with a solution of 3-n-butylphthalide or ligustilide in an amount sufficient to administer to a subject a daily dose of 100 mg of 3-n-butylphthalide or ligustilide per kg body weight. For an average cat of 5 kg of body weight to consume approx. 400 g of wet food per day, the cat food contains 1250 mg/kg.

### Example 16

### Dog Treats Containing 3-n-Butylphthalide or Ligustilide

Commercial dog treats (e.g. Mera Dog “Biscuit” for dogs as supplied by Mera Tiernahrung GmbH, Marienstrasse 80-84, 47625 Kevelaer-Wetten, Germany) are sprayed with a solution of 3-n-butylphthalide or ligustilide in an amount sufficient to administer to the treats 5-50 mg of 3-n-butylphthalide or ligustilide per g treats. The food composition is dried to contain dry matter of about 90% by weight.

### Example 17

### Cat Treats Containing 3-n-Butylphthalide or Ligustilide

Commercial cat treats (e.g. Whiskas Dentabits for cats as supplied by Whiskas, Master-foods GmbH, Eitzer Str. 215, 27283 Verden/Aller, Germany) are sprayed with a solution of 3-n-butylphthalide or ligustilide in an amount sufficient to administer to the treats 5-50 mg of 3-n-butylphthalide or ligustilide per g treats. The food composition is dried to contain dry matter of about 90% by weight.

1. A compound of the general formula I, 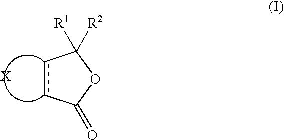
wherein
R¹ is hydrogen or hydroxy-;
R² is butyl or butyryl if R¹ is hydroxy, but R² is butyl if R¹ is hydrogen; or
R¹ and R² taken together are 1-propylidene or 1-butylidene optionally substituted by hydroxy, methyl, or 3-(α,β-dimethylacryloyloxy);
the dotted line is an optional bond;
X is an optionally substituted aliphatic C4-residue selected from the group consisting of X1, X2, X3, X4, and X5; 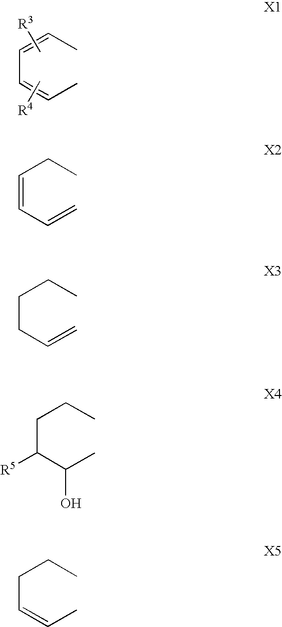
wherein
X is X2, X3 or X5 if the dotted line in formula (I) is absent; and
X is X1, X4 or X5 if the dotted line signifies a bond in formula (I) above;
R³ and R⁴ are, independently from each other, hydrogen or hydroxy; and
R⁵ is hydroxy or butyryl,
for use in the treatment of a disorder connected to impaired neurotransmission or for use in the preparation of compositions useful for the treatment of a disorder connected to impaired or reduced neurotransmission.

2. A compound for use as in claim 1, wherein the compound is selected from the group consisting of (E)-senkyunolide E; senkyunolide C; senkyunolide B; 3-butyl-4,5,6,7-tetrahydro-3,6,7-trihydroxy-1(3H)-isobenzofuranone; 3-butyl-1(3H)-isobenzofuranone; 3-butylphthalide; 3-butylidenephthalide; 3-propylidenephthalide; chuangxinol; ligustilidiol; senkyunolide F; 3-hydroxy-senkyunolide A; angeloylsenkyunolide F; senkyunolide M; 3-hydroxy-8-oxo-senkyunolide A; ligustilide; 6,7-dihydro-(6S,7R)-dihydroxyligustilide; 3a,4-dihydro-3-(3-methylbutylidene)-1(3H)-isobenzofuranone; sedanolide; and cnidilide, and mixtures thereof.

3. A compound for use as in claim 1, wherein the compound is selected from the group consisting of ligustilide, 3-butylphthalide, 3-butylidenephthalide, 3-propylidenephthalide, sedanolide, senkyunolide A, senkyunolide B and senkyunolide C and mixtures thereof.

4. A compound according to claim 1 wherein the compound is used as antidepressant, mood/vitality improver, stress reliever, condition improver, reducer of anxiety, reducer of obsessive-compulsive behaviour, relaxant, sleep improver and/or insomnia alleviator.

5. A dietary pharmaceutical composition containing a compound as defined in claim 1 for the treatment of a disorder connected to impaired or reduced neurotransmission.

6. The dietary composition according to claim 5 being in form of food in solid or liquid form such as soups or dairy products, in form of fortified food such as cereal bars and bakery items such as cakes and cookies, in form of dietary supplements such as tablets, pills, granules, dragées, capsules, and effervescent formulations, in form of non-alcoholic drinks such as soft drinks, sport drinks, fruit juices, lemonades, teas and milk based drinks.

7. A pharmaceutical composition according to claim 5 comprising conventional physiologically acceptable carriers, excipients or diluents.

8. The use of a compound of the general formula I as defined in claim 1 for the treatment of a disorder connected to impaired neurotransmission or for the manufacture of compositions useful for the treatment of a disorder connected to impaired neurotransmission.

9. The use of a compound according to claim 7 as antidepressant, mood/vitality improver, stress reliever, condition improver, reducer of anxiety, reducer of obsessive-compulsive behaviour, relaxant, sleep improver and/or insomnia alleviator.

10. The use of a dietary or pharmaceutical composition according to claim 5 as antidepressant, mood/vitality improver, stress reliever, condition improver, reducer of anxiety, reducer of obsessive-compulsive behaviour, relaxant, sleep improver and/or insomnia alleviator.

11. A method for the treatment of a disorder connected to impaired neurotransmission in animals including humans, said method comprising (a) preparing a composition according to claim 5 and (b) administering an effective dose of said composition to animals including humans which are in need thereof.

12. The method according to claim 11, wherein the animal is a human, a pet animal or a farm animal.

13. Use of a compound of claim 1 in the manufacture of a composition to inhibit serotonin, noradrenaline, or dopamine re-uptake by monomine transporters in pre-synaptic neurons.

14. The use according to claim 13, wherein the compound of the general formula I is selected from the group consisting of (E)-senkyunolide E; senkyunolide C; senkyunolide B; 3-butyl-4,5,6,7-tetrahydro-3,6,7-trihydroxy-1(3H)-isobenzofuranone; 3-butyl-1(3H)-isobenzofuranone; 3-butylphthalide; 3-butylidenephthalide; 3-propylidenephthalide; chuangxinol; ligustilidiol; senkyunolide F; 3-hydroxy-senkyunolide A; angeloylsenkyunolide F; senkyunolide M; 3-hydroxy-8-oxo-senkyunolide A; ligustilide; 6,7-dihydro-(6S,7R)dihydroxyligustilide; 3a,4-dihydro-3-(3-methylbutylidene)-1(3H)-isobenzofuranone; sedanolide; and cnidilide, and mixtures thereof.

15. The use according to claim 13, wherein the compound of the general formula I is selected from the group consisting of ligustilide, 3-butylphthalide, 3-butylidenephthalide, 3-propylidenephthalide, sedanolide, senkyunolide A, senkyunolide B and senkyunolide C, and mixtures thereof.

16. The use of a compound according to claim 13, wherein the animals are companion animals.

17. The use of a compound according to claim 13, wherein the animals are farm animals.

18. The use according to claim 17, wherein the farm animals are poultry.

19. The use according to claim 17, wherein the farm animals are selected from the group consisting of: cattle, sheep, goat and swine.

20. The use of a compound according to claim 13, which is reducer of tension, sadness, unhappiness, discontentedness, irritability or dysphoria.

21. The use of a compound according to claim 17, wherein the compound is used for preventing stress in farm animals in mass production lifestock husbandry, during transport to slaughter and/or for preventing quality loss of meat of said farm animals during transport to slaughter.

22. The use according to claim 18, wherein the compound of the general formula I is used for preventing loss in egg production, feather picking and cannibalism and losses of meat quality upon transport stress.

23. The use of a compound according to claim 13, for normalizing and maintaining the circadian rhythm in humans, or for alleviating or for preventing the symptoms associated with a disturbed circadian rhythm in humans.

24. The use according to claim 13, wherein the animals for the fur industry.

25. The use according to claim 16, wherein the use is selected from the group consisting of: reduction of stress, reduction of tension, reduction of aggressiveness, reduction of compulsive behavior, reduction of stress due to separation, change or loss of the owner, reduction of stress during holiday separation, and reduction of stress in “animal hotels”, reduction of stress in animal shelter stations, and reduction of stress in conditions of dense husbandry and breeding.
